(19) United States
(12) Patent Application Publication
(10) Pub. No.: US 2008/0044861 A1
(43) Pub. Date: Feb. 21, 2008

(54) Methods for Recovering Isoflavones from Fermentation Processes

(75) Inventor: J. Mark Weber, Chicago, IL (US)

Correspondence Address: THOMPSON COBURN, LLP, ONE US BANK PLAZA, SUITE 3500, ST LOUIS, MO, 63101, US

(73) Assignee: FERMALOGIC, INC., Chicago, IL (US)

(21) Appl. No.: 11/464,700

(22) Filed: Aug. 15, 2006

(51) Int. Cl. C12P 19/60; C12P 17/04; C12N 1/20

(52) U.S. Cl. 435/75; 435/252.35; 435/126

(57) ABSTRACT

A method for obtaining improved yields of isoflavones from spent fermentation beer produced by Actinomycetes grown on leguminous plant materials is demonstrated. This method involves conversion of isoflavone biotransformation products to isoflavone aglycones followed by isolation of the isoflavone aglycones. Formation of the biotransformation products had previously limited the production of isoflavone aglycones in these Actinomycete fermentations.

### STATEMENT REGARDING FEDERALLY SPONSORED RESEARCH OR DEVELOPMENT

This invention was made with Government support under a National Cancer Institute of the National Institute of Health Grant No. R44 CA093165. The government has certain rights to this invention.

### CROSS-REFERENCE TO RELATED APPLICATIONS

None.

### APPENDIX

Not Applicable.

BACKGROUND OF THE INVENTION

1. Field of the Invention

This invention relates generally to methods of producing isoflavones in fermentation processes and, more particularly, to methods of producing isoflavones in soy-based fermentation processes with actinomycetes.

2. Related Art

Microbial natural product manufacturing for the production of antibiotics, anti-cancer agents, immunosuppressants and other medically important compounds involves very large scale fermentation processes. A typical manufacturing process for a typical antibiotic may use a dozen 100,000 L (or more) fermentors dedicated to antibiotic production on a full time basis. The fermention process is completed in about 7-10 days. Many microbial natural product manufacturing processes utilize large quantities of soybean flour or grits. Each 100,000 L fermentor is filled with a nutrient broth that typically contains about 2.2 metric tonnes of soybean material per tank. The soybean material primarily serves as a source of carbon and nitrogen for growth of the fermentation organism in these processes.

Given the volume of material used in such fermentations, manufacturers of microbial natural products have sought to find some use for the spent fermentation beer that remains following the extraction of the desired natural product. For example, U.S. Pat. No. 6,616,953 describes compositions and methods for obtaining poultry feed additive from the spent fermentation beer obtained from fermentation of Saccharopolyspora erythraea. A process whereby the spent fermentation beer from Saccharopolyspora erythraea-mediated erythromycin production processes is first treated with acid and heat and then treated with a mixture of at least two enzymes is disclosed. The product of the acid, heat and enzyme treatments is then simply concentrated for use as poultry feed. The chemical composition of the concentrated end product of the process is not disclosed. U.S. Pat. No. 6,616,953 also does not disclose any steps or methods for isolating or further purifying any particular components from the concentrated product produced by the disclosed process.

The purification of distinct, high value chemical products from spent fermentation beer represents another approach to regaining value from fermentation processes. Leguminous plants such as soy are also a rich source of isoflavones, a group of valuable compounds that can confer a variety of health benefits when consumed (Kaufman et al., Journal of Alternative and Complementary Medicine, 3(1):7-12, 1997). Legumes typically contain isoflavone glucosides as well as the isoflavones daidzein and/or genistein. It is the aglycone forms of isoflavones such as daidzein or genistein, referred to here as isoflavones, that are the primary biologically active compounds that confer health benefits when consumed. Purified isoflavones can also be used as intermediates in the synthesis of potential derivatives that can be used as drugs. At present purified genistein can cost about $75 to $1,125 a gram.

The transient production of limited amounts of isoflavones from the erythromycin fermentation of Saccharopolyspora erythraea has been described (U.S. Pat. No. 5,554,519; Hessler et al., 1996). U.S. Pat. No. 5,554,519 and Hessler et al. show that during the initial stages of this fermentation process, isoflavone glucosides from the soybean meal are first converted into the desirable isoflavones, daidzein and genistein. However, a significant percentage of the desirable isoflavones were subsequently converted into other unidentified products by the end of the fermentation process. U.S. Pat. No. 5,554,519 estimated that about 25% of the initially available genistein was recovered by the disclosed process that comprised extracting the genistein from the fermentation media in an organic solvent under alkaline conditions. In this process, the genistein was co-purified with the antibiotic erythromycin in this initial alkaline organic extraction step. The biochemical fate of the remaining 75% of the initially available genistein was not disclosed by these references. Hessler et al. (Ibid) speculated that additional enzymatic biotransformations act on the genistein during fermentation and that the yield of genistein could be improved by inactivating the Saccharopolyspora erythraea genes that encode the biotransforming enzymes.

To increase the yield of the desirable isoflavone aglycones from fermentations, a number of undesirable, yield-limiting modifications must be inhibited. Various modifications of the desirable isoflavone aglycones by microorganisms have been described. For example, Chimura et al. (J. Antibiot. 28, 619-626, 1975) report that Streptomyces roseolus converts isoflavone aglycones to undesirable hydroxylated or methoxylated isoflavone derivatives. Undesirable hydroxylation, methylation, and even the chlorination of genistein by various Streptomycetes has also been described (Hosny and Rosazza, J. Nat. Prod. 62, 1609-1612, 1999; Komiyama, et al. J. Antibiotic 42, 1344-1349, 1989). Rhamnosylated derivatives of isoflavones are also known to be produced by various actinomycetes (Aoyagi, et al., J Antibiot (Tokyo), 28(12):1006-8; 1975; Hazato, et al., J. Antibiot. 32, 217-222, 1979). Microorganisms such as Nocardia species NRRL 5646 and Mortierella have also been shown to hydroxylate, methylate, and rhamnosylate daidzein (Maatooq and Rosazza, Phytochemistry 66(9):1007-11, 2005). In view of the many potential biotransformations of isoflavones that have been reported, improving yields of isoflavones from actinomycete fermentations by inactivating genes that encode the biotransforming enzymes clearly represents a difficult task. Given that undesirable isoflavone biotransformation reactions were well documented in certain microorganisms, it was also not clear that isoflavone biotransformation products could be efficiently converted back to isoflavones once the biotransformation reactions had occurred. Nonetheless, there is a clear need for improving the yield of isoflavones from microbial fermentation processes.

### SUMMARY OF THE INVENTION

It is in view of the above problems that the present invention was developed. The inventors have discovered that the isoflavone biotransformation products produced during the fermentation processes can be efficiently converted back to the desired isoflavones. The invention is thus a process for producing isoflavones from actinomycete fermentations of leguminous plant materials whereby the biotransformation products of isoflavones are converted into isoflavones and then further purified.

The invention described herein provides a method for producing an isoflavone in a fermentation process, comprising the steps of growing an actinomycete selected from the group consisting of Streptomyces and Saccharopolyspora in a growth medium comprising material from a leguminous plant, obtaining an isoflavone biotransformation product capable of being converted into an isoflavone from spent growth media, converting the thus obtained isoflavone biotransformation product to an isoflavone, and then isolating the isoflavone. The isoflavone produced by this method are isoflavones in their aglycone forms such as genistein or daidzein. To practice this method, the isoflavone biotransformation product is an isoflavone that is reversibly modified. Reversibly modified isoflavone biotransformation products include, but are not limited to, glycosylated isoflavones (i.e., isoflavone glycones) and isoflavones that are rhamnosylated. Reversibly modified isoflavone biotransformation products also include, but are not limited to, isoflavones that have been esterified. The isoflavone biotransformation product is not an irreversibly modified isoflavone. Examples of irreversibly modified isoflavone biotransformation products that are not used in this method include isoflavones that have been oxidized, reduced, or hydrolyzed or isoflavones that have been hydroxylated, methoxylated, methylated, or chlorinated.

In certain instances, fermentation process that is used herein to produce an isoflavone is also a fermentation process for producing a drug or a drug precursor. In other words, the fermentation process that is used to produce the drug or drug precursor can also be used to produce an isoflavone such that both a drug or drug precursor and an isoflavone are ultimately isolated from the same fermentation. The drug produced is selected from the group consisting of oleandomycin, actinomycin, avermectin, lasalocid, tetracenomycin, tetracycline, oxytetracycline, tylosin, raparnycin, daunorubicin, rifamycin, monensin and erythromycin. Particularly useful fermentation processes for producing the isoflavones are processes that also produce erythromycin.

To practice this method, the actinomycete is any one of Streptomyces or Saccharopolyspora. Preferred actinomycetes that can be used in this invention include, but are not limited to, Streptomyces rimosus, Streptomyces fradiae, Streptomyces hygroscopicus, Streptomyces cinnamonensis, Streptomyces peucetius, Saccharopolyspora erythraea, Streptomyces avermitilis, and Streptomyces glaucescens. One particularly actinomycete useful in practicing this method is Saccharopolyspora erythraea. One particularly useful strain of Saccharopolyspora erythraea is Saccharopolyspora erythraea ATCC1635. However, any actinomycete that primarily converts isoflavones to reversibly modified isoflavone biotransformation products can be used in this method.

In this method the actinomycete is fermented on growth media that contains material from a leguminous plant. The leguminous plant is selected from the group consisting of soybean (Glycine max), lupine (Lupinus species), fava bean, (Vicia faba), bean (Phaseolus species), clover (Trifolium species), lentil (Lens species), Baptisia (Baptisia species), kudzu (Pueraria species), pea (Pisum species), Vigna species, garbanzo bean (Cicer species), alfalfa (Medicago species), and psoralea (Psoralea corylifolia). Particularly useful growth medias are those derived from soybean (Glycine max).

The isoflavone biotransformation product is obtained from the spent growth media by any one of a variety of techniques familiar to those skilled in the art. One method of obtaining the isoflavone biotransformation product is to extract the spent growth medium with a polar or non-polar organic solvent which is immiscible with water to obtain a solvent fraction and an aqueous fraction, so that the aqueous fraction thus obtained will contain the isoflavone biotransformation product. One group of useful polar or non-polar organic solvent for practicing this method are C2-C10 organic solvents. Specific polar or non-polar organic solvents that can be used include, but are not limited to, 1-butanol, 2-butanol, t-butanol, pentanol, hexanol, heptanol, octanol, ether, ethyl acetate, tetrahydrofuran, hexane, heptane, octane, isohexane, diethylether, methyl ethyl ketone, diisopropylether propanol, isopropyl alcohol, isobutyl alcohol, butanol, ethyl acetate, acetonitrile, acetone, methylene chloride, chloroform, carbon tetrachloride, and mixtures thereof. Another method of obtaining the isoflavone biotransformation product is to capture the isoflavone biotransformation product from the spent growth medium on a solid phase sorbent and then eluting the isoflavone biotransformation product from that solid phase sorbent. Solid phase sorbents that can be used for this purpose include, but are not limited to, non-polar polystyrene sorbents, C18 sorbents, anion exchange resins or sorbents, divinylbenzene sorbents, polystyrene-divinylbenzene sorbents or modified divinylbenzene sorbents.

The obtained isoflavone biotransformation product is converted to an isoflavone by any procedure that efficiently provides for the desired conversion. While any amount of conversion of the obtained isoflavone biotransformation product to an isoflavone above background conversion levels constitutes conversion, preferably at least 50% or greater of the available biotransformation product is converted to at least one isoflavone. More preferably, at least 70% or greater conversion of the available isoflavone biotransformation product is converted to at least one isoflavone. Most preferably, at least 95% or greater conversion of the available isoflavone biotransformation product is converted to at least one isoflavone. One method of converting the obtained isoflavone biotransformation product to an isoflavone entails treating that obtained isoflavone biotransformation product with a sufficient amount of an acid and a sufficient degree of heat to result in conversion of said isoflavone biotransformation product to an isoflavone. Acids including, but not limited to, boric acid, benzoic acid, butyric acid, carbonic acid, citric acid, hydrobromic acid, hydrochloric acid, hydrofluoric acid, hydroiodic acid, lactic acid, malic acid, mandelic acid, nitric acid, propionic acid, sulfuric acid, oxalic acid, perchloric acid, phosphoric acid, phosphonic acid, pyrophosphoric acid, pyruvic acid, valeric acid, acetic acid and formic acid can be used to convert the obtained isoflavone biotransformation product to an isoflavone. In one particular embodiment of this method, the obtained isoflavone biotransformation product is treated with about 3N to 6N HCl at a temperature of about 50° C. to 80° C. for about 3 hours. In another embodiment of this method, the obtained isoflavone biotransformation product is treated with about 3N to 4 N HCl at about 80° C. for about 3.5 hours. However, it is understood that any number of different combinations of acid, acid concentrations and heat can be used to effect conversion of the obtained isoflavone biotransformation product to an isoflavone.

Another method of converting the obtained isoflavone biotransformation product to an isoflavone entails a first step where the obtained isoflavone biotransformation product is treated with an acid and heat followed by a second step where the acid and heat treated biotransformation product from the first step is treated with at least one enzyme selected from the group consisting of a cellulase, a hemicellulase, a pectinase, an arabinosidase, or any combination of those enzymes. In this method, the amount of acid and heat in the first step and the amount of enzyme in said second step are sufficient to result in conversion of said isoflavone biotransformation product to an isoflavone. Acids useful for the first step include, but are not limited to, boric acid, benzoic acid, butyric acid, carbonic acid, citric acid, hydrobromic acid, hydrochloric acid, hydrofluoric acid, hydroiodic acid, lactic acid, malic acid, mandelic acid, nitric acid, propionic acid, sulfuric acid, oxalic acid, perchloric acid, phosphoric acid, phosphonic acid, pyrophosphoric acid, pyruvic acid, valeric acid, acetic acid and formic acid. The obtained isoflavone biotransformation product is treated in said first step with acid sufficient to obtain a pH of about 3.5-5.0 at a temperature from about 80° C. to about 100° C. Either one enzyme or a mixture of enzymes is used in the second step. Alternatively, only one enzyme such as a cellulase, a hemi-cellulase, an arabinosidase or a pectinase is used in said second step. A mixture of cellulase, a hemi-cellulase, a pectinase and an arabinosidase can also be used in said second step. This method can further comprise the step of cooling the acid and heat treated biotransformation product from the first step to a temperature from about 40° C. to about 60° C. before adding at least one enzyme in a subsequent step.

All of the methods for producing an isoflavone described here can also comprise the step of concentrating the isoflavone biotransformation product prior to conversion of that biotransformation product to an isoflavone. Concentration of the obtained biotransformation product prior to its conversion can be effected by any method known to those skilled in this art. One concentration method entails performing a solid phase extraction of the isoflavone biotransformation product of step (b) and recovering said isoflavone biotransformation product in a concentrated form. This solid phase extraction is effected with a sorbent selected from the group consisting of non-polar polystrenes, C18, divinylbenzene, polystyrene-divinylbenzene and modified divinylbenzene. Alternatively, the concentration can be effected by extracting the growth medium with a polar or non-polar organic solvent which is immiscible in water to obtain a solvent fraction and an aqueous fraction, increasing the acidity of said aqueous fraction of step (b) to about pH 3, extracting this acidified fraction of with a solvent, evaporating this solvent and then recovering said isoflavone biotransformation product in a concentrated form. Combinations of concentration techniques (i.e., a solid phase extraction of the biotransformation product followed by a solvent extraction or the converse thereof) to effect more efficient concentration and/or purification of the biotransformation product prior to its conversion to an isoflavone are also contemplated by this invention.

The final step of the method for producing isoflavones comprises the step of isolating the isoflavone. Isolation of an isoflavone comprises any method where the isoflavone is either completely or partially separated from the other byproducts that result from the execution of the preceding steps of the method (i.e., growing of the actinomycete on the desired media, obtaining the isoflavone biotransformation product, and converting the isoflavone biotransformation product to an isoflavone). In this regard, methods where the isolated isoflavone is substantially free of saccharides are contemplated. One method of isolating the isoflavone comprises extracting the converted isoflavone from the previous step that is in an aqueous phase with a polar or non-polar solvent that is immiscible in water, wherein said isoflavone is recovered in the solvent phase. This solvent is selected from the group consisting of propanol, isopropyl alcohol, isobutyl alcohol, butanol, ethyl acetate, acetonitrile, acetone, methylene chloride, chloroform, carbon tetrachloride, and mixtures thereof. Alternatively, the isolation of said isoflavone can comprise performing a solid phase extraction of the converted isoflavone from the previous step. This solid phase extraction can be effected with a sorbent selected from the group consisting of C18, divinylbenzene, polystyrene-divinylbenzene, non-polar polystyrene, anion exchange resin, and modified divinylbenzene. This sorbent can also be an anion exchange resin, where the isoflavone is released by treating said resin with an alcohol, an organic solvent, an admixture of alcohol and water, or an admixture of an organic solvent and water. Combinations of isolation techniques (i.e., a solid phase extraction of the biotransformation product followed by a solvent extraction or the converse thereof) to effect more efficient isolation or purification of the converted isoflavone are also contemplated by this invention.

Another method for producing an isoflavone in a fermentation process is also described herein. This alternative method of producing an isoflavone comprises the steps of growing a Streptomycete selected from the group consisting of Streptomyces antibioticus, Streptomyces griseus, Streptomyces lasaliensis, and Streptomyces parvulus in a growth medium comprising material from a leguminous plant; obtaining an isoflavone from a spent growth medium from the previous step, and isolating the isoflavone from spent growth media, thereby producing an isoflavone in a fermentation process. In this case, the isoflavone is obtained in the spent growth media by simply harvesting that spent growth media either as a cell lysate or as a cell free liquid. The isoflavone can be isolated from that liquid as per any of the solvent or solid phase extraction techniques described herein for isolation of isoflavones from liquids.

Further features and advantages of the present invention, as well as the structure and operation of various embodiments of the present invention, are described in detail below with reference to the accompanying drawings.

### BRIEF DESCRIPTION OF THE DRAWINGS

The accompanying drawings, which are incorporated in and form a part of the specification, illustrate the embodiments of the present invention and together with the description, serve to explain the principles of the invention. In the drawings:

FIG. 1 illustrates the first biotransformation of isoflavone glucosides into isoflavone aglycones during the Actinomycete fermentations.

FIG. 2 illustrates a thin layer chromatograph showing evidence of biotransformation of isoflavones during the erythromycin fermentation.

FIG. 3 illustrates the biotransformation of isoflavone aglycones into isoflavone rhamnosides that occurs in Actinomycete fermentations.

FIG. 4A, B, and C illustrate a comparison of strong acid (4N HCl) and heat treatment (80° C.) to treatment for the recovery of isoflavone aglycones. Panel D illustrates a comparison of Validase® BG (an endo beta 1,4 glucanase) and Crystalzyme® PML-MX treatments.

FIG. 5 illustrates the TLC analysis of additional Actinomycete fermentation products after growth in OFM1 soy media (minus oil) before and after strong acid and heat treatment.

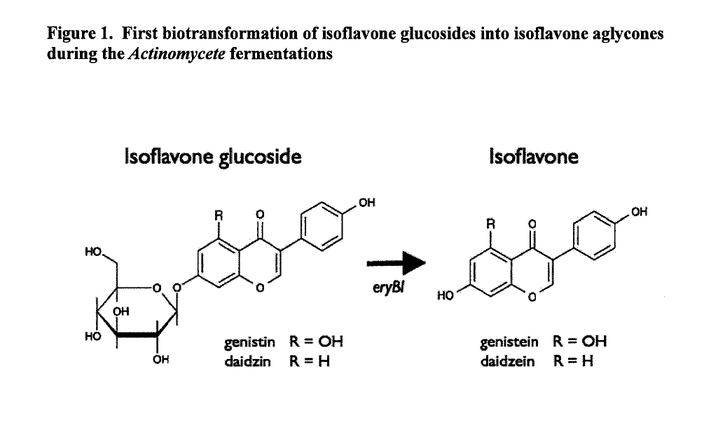

### DETAILED DESCRIPTION OF THE PREFERRED EMBODIMENTS

The following detailed description of the invention is provided to aid those skilled in the art in practicing the present invention. Even so, the following detailed description should not be construed to unduly limit the present invention as modifications and variations in the embodiments discussed herein may be made by those of ordinary skill in the art without departing from the spirit and scope of the present invention.

### Definitions

### The following words and phrases have the meanings set forth below.

Actinomycetes: As used herein, Actinomycetes refer to the group of gram-positive bacteria (order Actinomycetales) that produce various bioactive agents including antibiotics, enzymes, and vitamins. Examples of actinomycetes include Streptomyces and Saccharopolyspora species.
Biotransformation: As used herein, this term refers to any chemical change in a given compound that is a result of exposing that compound to a microbial growth medium.
Drug precursor: As used herein, a drug precursor is any compound produced in a fermentation process that is used in any subsequent processes to produce a drug or is any compound that is a pro-drug. A drug precursor can be a compound that is a drug (i.e., erythromycin) that is converted to other derived drugs by other subsequent processes.
Isoflavone: As used herein, the term “isoflavone” refers to the aglycone forms of the plant derived flavonoid compounds genistein, daidzein, biochanin, formononetin, and glycitein.
Isoflavone biotransformation product: As used herein, an isoflavone biotransformation product is any covalently modified form of an isoflavone that is produced when an micro-organism is cultured in the presence of an isoflavone.
Isolating an isoflavone: As used herein, the phrase “isolating an isoflavone” refers to any process whereby an isoflavone is separated from another component that is mixed with the isoflavone.
Performing a solid phase extraction: As used herein, the phrase “performing a solid phase extraction” describes any method whereby a sorbent is used to effect the extraction of a desired product from a liquid. Solid phase extraction methods can comprise execution of any one of, or all of, such steps as conditioning the sorbent, adsorbing the desired product (i.e., either an isoflavone biotransformation product or isoflavone) to the sorbent, washing the adsorbed material to remove contaminants, and eluting the product from the sorbent.
Pro-drug: As used herein, a pro-drug is any compound that is converted to an active derivative compound upon administration to a subject.
Reversibly modified isoflavone biotransformation product: As used herein, a reversibly modified isoflavone biotransformation product is a covalently modified isoflavone capable of being converted back into an isoflavone by the methods described herein. Examples of reversibly modified isoflavone biotransformation products include glycosylated isoflavones and esterified isoflavones.
Irreversibly modified isoflavones: As used herein, an irreversibly modified isoflavone biotransformation product is an isoflavone biotransformation product that cannot be converted back into an isoflavone by the methods described herein. Examples of irreversibly modified isoflavones include isoflavones that have been oxidized, reduced, hydrolyzed, hydroxylated, methoxylated, methylated or chlorinated.
Isoflavone glucoside biotransformation reaction: As used herein, the phrase isoflavone glucoside biotransformation reaction refers to the hydrolysis of an isoflavone glucoside to form an isoflavone. Examples of this biotransformation reaction include the hydrolysis of the isoflavone glucosides genistin and daidzin to form the isoflavone aglycones genistein and daidzein.

### Description of Suitable Microorganisms and Fermentation Conditions for Use in the Invention

A variety of microorganisms drawn from the order of gram positive Actinomycetes are useful in the practice of this invention. Certain Actinomycetes are characterized herein as having the ability to produce reversibly modified isoflavone biotransformation products in fermentations and are favored for use in this invention. Streptomycetes or Saccharopolyspora species are particularly favored sources of microorganisms for use in the methods of this invention. Actinomycetes that can reversibly modify isoflavone biotransformation products include Streptomyces rimosus, Streptomyces fradiae, Streptomyces hygroscopicus, Streptomyces cinnamonensis, Streptomyces peucetius, Saccharopolyspora erythraea, Streptomyces avermitilis, and Streptomyces glaucescens.

However, it is further understood that the use of Actinomycetes other than those specifically listed here that can reversibly modify isoflavone biotransformation products is also contemplated herein. In particular, it is envisioned that one of ordinary skill in the art could simply test any number of different Actinomycetes by performing the methods described herein to identify other Actinomycetes that have the ability to produce reversibly modified isoflavone biotransformation products.

Another common feature of the Actinomycetes used in the practice of this invention is that they are capable of converting the isoflavone glucosides that are present in the legume derived plant material in the growth media to isoflavones (i.e., the aglycone form). This initial isoflavone glucoside biotransformation reaction that occurs in certain Actinomycete fermentations has been described (U.S. Pat. No. 5,554,519; Hessler et al., 1996).

It is further recognized that certain microorganisms produce irreversibly modified isoflavone glucosides that cannot be converted to isoflavones. Examples of such irreversibly modified isoflavones include isoflavones that have been methylated, methoxylated, hydroxylated or chlorinated. Microorganisms that catalyzed one or more of the irreversible isoflavone biotransformations include Nocardia species and Mortierella isabellina (Maatooq and Rosazza, Phytochemistry 66(9):1007-1011, 2005). The use of microorganisms that catalyze irreversible biotransformations of isoflavones is not favored in the practice of this invention.

A key feature of the method is the use of growth media that contains material from leguminous plants. Leguminous plants are known to contain isoflavones and isoflavone glucosides (see Kaufman et al., Journal of Alternative and Complementary Medicine, 3(1):7-12, 1997). As described here, the certain microorganisms will produce reversibly modified isoflavone biotransformation products that can be converted to isoflavones when cultured on growth media that contains, amongst other ingredients, material from leguminous plants. Leguminous plants that can serve as a source of isoflavone glucosides and isoflavones include soybean (Glycine max), lupine (Lupinus species), fava bean, (Vicia faba), bean (Phaseolus species), clover (Trifolium species), lentil (Lens species), Baptisia (Baptisia species), kudzu (Pueraria species), pea (Pisum species), Vigna species, garbanzo bean (Cicer species), alfalfa (Medicago species), and psoralea (Psoralea corylifolia). A variety of distinct parts of the leguminous plant can be used to obtain the material used in the growth media. For example, the material used in the growth media can be derived from the leaves, stems, roots, seed pods, or seeds of the leguminous plant, or from a combination of those plant parts. Stover, or plant leaves and stems left in the field post-harvest, is another potential source of leguminous plant material that could be used in the growth media. In particular embodiments of the invention, soybean grits, soybean flour or soybean meal is used as the leguminous plant material in the fermentation.

Any type of fermentation vessel can be used to practice this invention. Fermentation vessels aerated by shaking, stirring or any other result effective method that provides for growth of the Actinomycete and efficient production of important fermentation products such as drugs and reversibly modified isoflavone biotransformation products can be used. The large 100,000 L fermentation vessels typically used by in batch mode production of antibiotics by microorganisms are particularly contemplated by this invention.

### Methods of Obtaining an Isoflavone Biotransformation Product from Spent Growth Media

The method of obtaining the reversibly modified biotransformation product for conversion is typically tailored to permit separation of the isoflavone biotransformation product from other desired fermentation products such as drugs. This also permits the subsequent conversion reactions that may be deleterious to the other fermentation products to be focused on the reversibly modified biotransformation product. Separation of the isoflavone biotransformation product from other desired fermentation products is also important as it can reduce or eliminate contamination of the isoflavones that are ultimately produced by the methods of this invention. In some instances, the Actinomycetes cells in the fermentation are simply lysed in the fermentation broth to obtain the spent growth media that contains the reversibly modified isoflavone biotransformation product. In other instances, the Actinomycetes cells in the fermentation are removed by techniques including, but not limited to, sedimentation, centrifugation or filtration to obtain the spent growth media that contains the reversibly modified isoflavone biotransformation product. Spent growth media obtained either by lysing the cells in the fermentation media or by separating the cells from the fermentation media can be used in the methods described herein. Finally, spent fermentation beer that contains whole Actinomycete cells can also be used.

A useful property of the reversibly modified biotransformation products is that they are polar and will remain in the aqueous phase when extracted with polar or non-polar solvents. Consequently, one method of obtaining these biotransformation products is to extract the spent growth medium with a polar or non-polar organic solvent which is immiscible with water to obtain an aqueous fraction that contains the reversibly modified isoflavone biotransformation product. In certain instances, such as in fermentations that produce erythromycin or other non-polar drugs, these drugs will be partitioned into the organic phase of that solvent extraction and thus separated from the reversibly modified isoflavone biotransformation product. The polar or non-polar organic solvent can be C2-C10 organic solvent. This polar or non-polar organic solvent can also be selected from the group consisting of 1-butanol, 2-butanol, t-butanol, pentanol, hexanol, heptanol, octanol, ether, ethyl acetate, tetrahydrofuran, hexane, heptane, octane, isohexane, diethylether, methyl ethyl ketone, diisopropylether propanol, isopropyl alcohol, isobutyl alcohol, butanol, ethyl acetate, acetonitrile, acetone, methylene chloride, chloroform, carbon tetrachloride, and mixtures thereof.

Solid phase extraction is another method for obtaining isoflavone biotransformation products from the spent growth media. Solid phase extraction of the isoflavone biotransformation products from the treated (i.e., converted) material can be accomplished by any number of different methods of solid phase extraction such as column-based methods or batch-based methods. The solid phase extraction is effected with a sorbent selected from the group consisting of C18, divinylbenzene, polystyrene-divinylbenzene, non-polar polystyrene, anion exchange resin, and modified divinylbenzene. Once the isoflavone biotransformation product is adsorbed to the solid phase sorbent, the adsorbed material is typically washed with aqueous solutions that do not displace the isoflavone biotransformation product from the sorbent to remove other undesired or contaminating materials. Alternatively, certain admixtures of alcohol and water that do not displace the isoflavone biotransformation product from the sorbent, or admixtures of an organic solvent and water that do not displace the isoflavone biotransformation product from the sorbent, can be used to remove other undesired or contaminating materials. Once the adsorbed material containing the isoflavone biotransformation product has been washed, the isoflavone biotransformation product can be released by using either an alcohol, an organic solvent, an admixtures of alcohol and water that displaces the isoflavone biotransformation product from the sorbent, or admixtures of an organic solvent and water that displace the isoflavone biotransformation product from the sorbent.

One method of effecting the solid phase purification of an isoflavone biotransformation product uses an anion exchange resin and releases the isoflavone(s) by treating said resin with an alcohol, an organic solvent, an admixture of alcohol and water, or an admixture of an organic solvent and water. Similar methods have been used to effect purification of glycosylated isoflavones or isoflavone conjugates from plant materials (U.S. Pat. Nos. 6,703,051 and 6,020,471) and can be adapted to the purification of the isoflavone biotransformation products obtained from the spent fermentation beer. In practicing this method, the anionic exchange resin is typically conditioned prior to use by first converting the resin to a hydroxide form, next converting the resin to a chloride or sulfate form, and then finally converting at least some strong base sites of the resin to a carbonate form. Useful anionic exchange resins include type II macroporous strong base anion exchange resin as well as weak base anion exchange resins. Type II strong base anion exchange resin comprise a quaternary ammonium type of resin in which the four substituents of the nitrogen atom are an ethanol group, two methyl groups, and a polymeric benzyl group. Useful type II strong base anion exchange resins include IRA 910 available from Rohm & Haas, Independence Mall West, Philadelphia, Pa. 19105; Dowex 22 available from Dow Chemical U.S.A., 2040 Willard H. Dow Center, Midland, Mich. 48674; and Ionac A651 available from Sybron, Sybron Chemical Division, Birmingham Road, Birmingham, N.J. 08011. Useful weak base anion exchange resins include Duolite A-7 from Rohm & Haas. The isoflavone biotransformation product is adsorbed to the conditioned anionic exchange resin, washed with an aqueous solution, and then eluted with an alcohol, an organic solvent, an admixture of alcohol and water, or an admixture of an organic solvent and water. Useful alcohols or organic solvents for elution of the isoflavone biotransformation product from the anion exchange resin include, but are not limited to, methanol, ethanol, propanol, isopropyl alcohol, isobutyl alcohol, butanol, ethyl acetate, acetonitrile, acetone, aqueous mixtures of the foregoing solvents, methylene chloride, chloroform, carbon tetrachloride, or a mixture of any of the foregoing solvents.

Other methods, including those found in U.S. Pat. No. 5,679,806, for the solid phase extraction of glycosylated isoflavones from plant materials can also be adapted to the solid phase extraction of isoflavone biotransformation products.

### Methods of Converting Isoflavone Biotransformation Products to Isoflavones

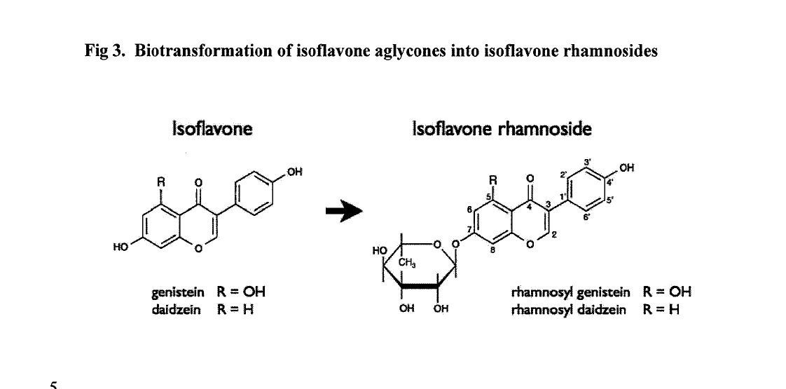

Having obtained reversibly modified isoflavone biotransformation products, the next step of the method of the invention is to convert that product to an isoflavone (i.e., an isoflavone aglycone). Without being limited by theory, the reversibly modified isoflavone biotransformation products are believed to be isoflavone derivatives or conjugates that are glycosylated or esterified. Preferably, the reversibly modified isoflavone biotransformation products that are O-glycosylated or esterified at some or all of the available positions (i.e., C-7, and C-4′ for daidzein, and C-7, C-4′ and C-5 for genistein). It has been determined that one of the primary isoflavone biotransformation products produced in Saccharopolyspora fermentations is an isoflavone rhamnoside (FIG. 3). However, this isoflavone rhamnoside is apparently converted to more complex reversibly modified isoflavone biotransformation products in Saccharopolyspora fermentations. Although the exact structure of the reversibly modified isoflavone biotransformation products in Streptomyces or Saccharopolyspora fermentations has not been elucidated, it has nonetheless been discovered that these products can be converted into the more desirable isoflavone products (i.e., isoflavone aglycones).

In the context of this invention, conversion of a reversibly modified isoflavone biotransformation product to an isoflavone comprises the conversion of any amount of the reversibly modified isoflavone biotransformation product to any isoflavone. In this context, “any amount of conversion” is taken to be an amount of conversion that would exceed the amount of “background” conversion of reversibly modified isoflavone biotransformation products that might occur in a spent fermentation broth or derived reversibly modified isoflavone biotransformation product sample held at up to 40° C. at pH3 or greater. Preferably, at least 50% or greater of the available (i.e., convertible) reversibly modified isoflavone biotransformation product is converted to an isoflavone. The amount of available (i.e., convertible) reversibly modified isoflavone biotransformation product can be determined by applying any one of the conversion methods described here, removing aliquots at various time points, and determining the time point at which the maximal amount of conversion of isoflavone biotransformation product to isoflavones has occurred. This maximal amount of conversion thus defines the amount of available (i.e., convertible) reversibly modified isoflavone biotransformation product. For example, the isoflavone biotransformation product is treated with about 3N to 4N HCl at about 80° C. for about 1, 2, 3, 4 and 5 hours to determine the maximal amount of conversion of reversibly modified isoflavone biotransformation product to isoflavones. The amount of conversion can be determined by thin-layer chromatography or any other suitable combination of separative (i.e., HPLC) and analytic techniques (UV absorption, fluorescence, Evaporative Light Scattering, mass spectrometry), where purified isoflavones (i.e., known amounts of genistein, daidzein, biochanin, formononetin, and/or glycitein) are used as qualitative and quantitative standards. In more preferred embodiments, 70% or greater of the available (i.e., convertible) reversibly modified isoflavone biotransformation products are converted to an isoflavones. In the most preferred embodiments, 95% or more of the available reversibly modified isoflavone biotransformation products are converted to an isoflavones.

One method of converting an isoflavone biotransformation product to an isoflavone is to treat the isoflavone biotransformation product with a sufficient amount of an acid and a sufficient degree of heat to result in conversion of said isoflavone biotransformation product to an isoflavone. In the context of this invention, sufficient acid and sufficient heat are defined as the amount and acid and heat required to convert any amount of the reversibly modified isoflavone biotransformation product to any isoflavone. In this context, “any amount of conversion” is taken to be an amount of conversion that would exceed the amount of “background” conversion of reversibly modified isoflavone biotransformation products that might occur in a spent fermentation broth or derived reversibly modified isoflavone biotransformation product sample held at up to 40° C. at pH3 or greater. Preferably, at least 50% of the available reversibly modified isoflavone biotransformation products are converted to isoflavones. As noted above, conversion of 70% or greater of the available reversibly modified isoflavone biotransformation products to isoflavones is more preferred, and a 95% or greater conversion rate is most preferred. Sufficiency of the acid and heat treatment are easily determined by comparing the percent conversion that is observed in the test acid and heat conditions to the amount of conversion that occurs in the conversion methods taught herein (i.e., treatment of the reversibly modified isoflavone biotransformation product with about 3N to 6N HCl at a temperature of about 50° C. to 80° C. for about 3 hours or with about 3N to 4N HCl at about 80° C. for about 3.5 hours). A variety of acids can be used to effect this conversion. For example, boric acid, benzoic acid, butyric acid, carbonic acid, citric acid, hydrobromic acid, hydrochloric acid, hydrofluoric acid, hydroiodic acid, lactic acid, malic acid, mandelic acid, nitric acid, propionic acid, sulfuric acid, oxalic acid, perchloric acid, phosphoric acid, phosphonic acid, pyrophosphoric acid, pyruvic acid, valeric acid, acetic acid and formic acid can be used in the conversion reaction. Another variable in the conversion method is time. A period of time sufficient to complete the conversion reaction in the presence of sufficient heat and acid is used. As described above, time course determinations would permit one skilled in the art to determine if sufficient time has been allowed for conversion of 50% or greater of the available reversibly modified isoflavone biotransformation products.

Another method of converting the obtained isoflavone biotransformation product to an isoflavone entails a first step where the obtained isoflavone biotransformation product is treated with an acid and heat followed by a second step where the acid and heat treated biotransformation product from the first step is treated with at least one enzyme selected from the group consisting of a cellulase, a hemicellulase, a pectinase, an arabinosidase, or any combination of those enzymes. In this method, the amount of acid and heat in the first step and the amount of enzyme in said second step are sufficient to result in conversion of said isoflavone biotransformation product to an isoflavone. Sufficiency of the amount of acid, heat, and enzyme treatment are defined as the amount and acid, heat and enzyme required to convert any amount of the reversibly modified isoflavone biotransformation product to any isoflavone In this context, “any amount of conversion” is taken to be an amount of conversion that would exceed the amount of “background” conversion of reversibly modified isoflavone biotransformation products that might occur in a spent fermentation broth or derived reversibly modified isoflavone biotransformation product sample held at up to 40° C. at pH3 or greater. Preferably, at least 50% of the available reversibly modified isoflavone biotransformation products are converted to isoflavones. Conversion of 70% or greater of the available reversibly modified isoflavone biotransformation products to isoflavones is more preferred, and a 95% or greater conversion rate is most preferred. As previously described, the amount of available (i.e., convertible) reversibly modified isoflavone biotransformation product can be determined by applying any one of the conversion methods described here (i.e., acid and heat or acid and heat followed by one or more enzymes), removing aliquots at various time points, and determining the point at which the maximal amount of conversion of biotransformation product to isoflavones has occurred.

Acids useful for the first step include, but are not limited to, boric acid, benzoic acid, butyric acid, carbonic acid, citric acid, hydrobromic acid, hydrochloric acid, hydrofluoric acid, hydroiodic acid, lactic acid, malic acid, mandelic acid, nitric acid, propionic acid, sulfuric acid, oxalic acid, perchloric acid, phosphoric acid, phosphonic acid, pyrophosphoric acid, pyruvic acid, valeric acid, acetic acid and formic acid. The obtained isoflavone biotransformation product is treated in said first step with acid sufficient to obtain a pH of about 3.0-5.0 at a temperature from about 80° C. to about 100° C. Either one enzyme or a mixture of enzymes is used in the second step. Alternatively, only one enzyme such as a cellulase, a hemi-cellulase, or a pectinase is used in said second step. A mixture of cellulase, a hemi-cellulase, a pectinase and an arabinosidase can also be used in said second step. As used here, a cellulase can comprise either an exo-beta-1,4-glucanase activity (i.e., a cellulase that preferentially hydrolyses terminally linked sugars of polysaccharides) or a combination of both an exo-beta-1,4-glucanase activity and an endo beta-1,4-glucanase activity (i.e., enzymes that hydrolyze interior beta-1,4-bonds in polysaccharides to release dextrins and oligosaccharides from beta-glucans). Beta glucanases that comprise an endo beta-1,4-glucanase activity (i.e., enzymes that hydrolyze interior beta-1,4-bonds in polysaccharides to release dextrins and oligosaccharides from beta-glucans) cannot be used as a sole enzyme in this method to effect conversion of the reversibly modified isoflavone biotransformation products. However, a beta glucanase comprising an endo beta-1,4-glucanase activity can be used in the second step of this method with any one of a cellulase comprising an exo-beta-1,4-glucanase activity, a hemi-cellulase, a pectinase or an arabinosidase. Alternatively, a beta glucanase comprising an endo beta-1,4-glucanase activity can be used in the second step of this method in combination with a hemi-cellulase, a pectinase or an arabinosidase. As used here, a hemicellulase is any enzyme that catalyzes the degradation or hydrolysis of hemicellulose. Examples of types of hemicellulose include but are not limited to xylan, glucuronoxylan, arabinoxylan, glucomannan, and xyloglucan. As used herein, a pectinase is any enzyme that catalyzes the degradation or hydrolysis of pectin. Pectins include, but are not limited to, homogalacturonan, rhamnogalacturonan I (which comprises alternating L-rhamnose and D-galacturonic acid subunits with α-(1-5)-L-arabinan and β-(1-4)-D-galactan side chains), and Rhamnogalacturonan II. Pectinases include, but are not limited to, polygalacturonase enzymes and rhamnogalacturonidase enzymes. As used herein, an arabinosidase is any enzyme that degrades or hydrolyses an arabinan, an arabinoxylan, or an arabinogalactan. Arabinosidases include, but are not limited to, arabinan endo-1,5-alpha-L-arabinosidases, beta-L-arabinosidases, and alpha-N-arabinofuranosidases. As used herein, an arabinase is an arabinan endo-1,5-alpha-L-arabinosidase.

Another variable in the two step conversion method using acid and heat and then one or more enzyme(s) is time. A period of time sufficient to complete the conversion reaction in the presence of sufficient heat and acid (in the first step) and then in the presence of sufficient enzyme(s) is used. As described above, time course determinations would permit one skilled in the art to determine if sufficient time has been allowed for conversion of 50% or greater of the available reversibly modified isoflavone biotransformation products.

This method can further comprise the step of cooling the acid and heat treated isoflavone biotransformation product from the first step to a temperature from about 40° C. to about 60° C. before adding at least one enzyme in a subsequent step. Conversion of 80% or greater of the available reversibly modified isoflavone biotransformation products to isoflavones is preferred, and a 95% or greater conversion rate is most preferred. However, the use of thermostable enzymes (i.e., enzymes that are active at temperatures of greater than 60° C.) is further contemplated. Thermostable enzymes are typically obtained from thermophilic organisms or by mutagenesis and selection of thermostable variants of enzymes. Use of thermostable enzymes could obviate completely or lessen the amount of cooling necessary after completion of the first step of this method.

Furthermore, the acid and heat treated biotransformation product from the first step can also be adjusted to a pH that matches the pH optima of the enzyme or enzymes used in the second step. This is accomplished by neutralizing the acid present in the sample containing the acid and heat treated isoflavone biotransformation product with either an appropriate base or an appropriate buffer. Appropriate bases include, but are not limited to, sodium, ammonium or potassium hydroxide. Appropriate buffers include, but are not limited to, Tris buffers, sodium phosphate buffers, sodium citrate buffers and sodium succinate buffers.

### Methods of Isolating Isoflavones

Having converted the reversibly modified isoflavone biotransformation product to an isoflavone, the final step of the claimed method entails isolation of the isoflavone from other compounds present in the treated (i.e., converted) material that has been subjected to any of the previously described conversion methods. As defined earlier, “isolating an isoflavone” refers to any process whereby an isoflavone is separated from another component that is mixed with the isoflavone. In this regard, isolation of the isoflavone may be either partial or complete. In other words, isolation of the isoflavone may represent nothing more than a partial purification whereby the isoflavone present in the converted material is separated from at least one component that is present in the original mixture. For example, it is anticipated that various saccharides (i.e., mono-, di-, oligo-, and poly-saccharides such as glucose, fructose, rhamnose, sucrose, cellulose, etc.) will be present in the treated (i.e., converted) material that also contains the isoflavone that has been released from the reversibly modified biotransformation products. Any step or method, such as a solvent extraction, that separates the isoflavone from those saccharides would thus constitute an isolation of an isoflavone in the context of this invention, even though other plant compounds that happen to copurify with the isoflavone are also present in the fraction that contains the isoflavone. The isolated isoflavone obtained by this method would thus be substantially free of saccharides. In this regard, it is possible that the isolated isoflavone, though substantially free of some compound originally present in the treated (i.e., converted) mixture, can also contain other compounds such as flavanoids, flavones, or flavanones.

Concentration of an isoflavone such that the isoflavone is not separated from any other component that is mixed with the isoflavone does not constitute “isolating an isoflavone”. For example, if a treated (i.e., converted mixture) containing the isoflavone released from the reversibly modified biotransformation products and other compounds is simply subjected to a distillation or evaporation step such that all of the components originally present in that mixture are still present in roughly the same ratios (i.e., molar ratios or mass ratios), then this distillation or evaporation step would not constitute an isolation step as described herein.

Of course, isolation of an isoflavone can also comprise further purification of one or more isoflavones from the treated (i.e., converted mixture) from the previous step of the method of this invention. For example, any one of the isoflavones produced by the method of this invention (i.e., genistein, daidzein, biochanin, formononetin, or glycitein) can be purified to a level of at least about 70%, 80%, 90%, 95% or 99% or greater purity on a mass basis using methods described herein or known to those of skill in the art. Alternatively, two isoflavones, such as genistein, daidzein, can be co-purifed to a level of at least about 70%, 80%, 90%, 95% or 99% or greater purity on a mass basis using methods described herein or known to those of skill in the art.

A variety of methods for isolating isoflavones can be used. Particularly contemplated are methods involving solvent extraction and solid phase extraction with sorbents. It is of course understood that a combination of isolation methods can also be used, especially when isolating isoflavones in more purified forms. It is also understood that other techniques, such as preparative column chromatography or high-performance liquid chromatography can be used either alone or in combination with methods such as solid phase or solvent extraction to isolate isoflavones from the treated mixture.

With respect to isolation of isoflavones by solvent extraction of the treated (i.e., converted material), isolation of the isoflavone can be effected by extracting said isoflavone from an aqueous phase comprising the treated material with a polar or non-polar solvent that is immiscible in water and recovering the isoflavone in the solvent phase. This solvent can be propanol, isopropyl alcohol, isobutyl alcohol, butanol, ethyl acetate, acetonitrile, acetone, methylene chloride, chloroform, carbon tetrachloride, or a mixtures of these solvents. Once the isoflavone is isolated in the solvent, it can be recovered by evaporating the solvent.

A variety of methods for extracting isoflavones from plant tissue with solvents have been described that can also be adapted for use in this particular method for isolating isoflavones from treated (i.e., converted) materials obtained from reversibly modified biotransformation products produced in fermentations. For example, U.S. Pat. No. 7,015,339 describes a method for isolating phenolic compounds such as isoflavone aglycones comprising the steps of: (a) providing an aqueous plant extract at a first pH greater than 10, the aqueous plant extract comprising a plurality of phenolic compounds; (b) washing the aqueous plant extract with an organic solvent; (c) adjusting the pH of the aqueous plant extract to a pH of less than 9; and (d) isolating the phenolic compounds from the aqueous plant extract. In the context of this invention, this method of U.S. Pat. No. 7,015,339 can be adapted to provide for isolation of isoflavones by adjusting the aqueous treated (i.e., converted) material containing the isoflavone aglycones to a pH greater than 10, washing with an organic solvent, adjusting the aqueous phase to a pH of less than 9, and then isolating the isoflavones from the aqueous fraction. That same patent also provides a similar method for isolating plant phenolics that include isoflavone aglycones comprising (a) providing an aqueous plant extract at a first pH less than 10, the aqueous plant extract comprising a plurality of phenolic compounds; (b) extracting the aqueous plant extract with a first organic solvent to yield a first organic extract; (c) extracting the first organic extract with an aqueous phase of pH greater than 10 to yield a phenol rich aqueous phase; (d) adjusting the pH of the phenol rich aqueous phase to a pH of less than 9; and (e) isolating the phenolic compounds from the phenol rich aqueous phase. In the context of this invention, this method of U.S. Pat. No. 7,015,339 can be adapted to provide for isolation of isoflavone aglycones from the aqueous treated (i.e., converted) material by providing the aqueous treated (i.e., converted) material at a pH of less than 10, extracting with an organic solvent to yield a first organic solvent containing the isoflavone aglycone, and then extracting that organic solvent phase with an aqueous phase of pH greater than 10 to yield an aqueous phase containing the isoflavones, adjusting this aqueous phase back to a pH of less than 9, and then isolating the isoflavones from that aqueous phase. Other examples of methods for isolating isoflavones from plant materials or other sources with solvents that can be readily adapted and applied to isolating the isoflavones produced by treatment of spent fermentation beer are described in U.S. Pat. Nos. 5,554,519 and 6,517,840.

U.S. Pat. No. 7,033,621 is especially notable in that it describes a process for the production of isoflavones from plants which comprises simultaneously contacting plant material with water, an enzyme which cleaves isoflavone glycosides to the aglucone form, and a C.sub.2-C10 organic solvent, to form a combination and incubating the combination for a time sufficient to allow isoflavones of the aglucone form to partition into the organic solvent component, and thereafter recovering isoflavones from the organic solvent component. This method could be adapted to the methods of this invention where the conversion step comprises treatment with an acid and heat followed by an enzyme treatment by coincubating the aqueous material containing the enzyme(s) with the organic solvent. The isoflavones can then be isolated from the organic solvent fraction at the conclusion of the reaction. The advantage of this particular process is that it represents a “one pot, single stage” process that can simplify the isoflavone isolation procedure.

Solid phase extraction is another method for isolating isoflavones from the treated (i.e., converted) material produced by this method. Solid phase extraction of the isoflavones from the treated (i.e., converted) material can be accomplished by any number of different methods of solid phase extraction such as column-based methods or batch-based methods. The solid phase extraction is effected with a sorbent selected from the group consisting of C18, divinylbenzene, polystyrene-divinylbenzene, non-polar polystyrene, anion exchange resin, and modified divinylbenzene. Once the isoflavone is adsorbed to the solid phase sorbent, the adsorbed material is typically washed with aqueous solutions that do not displace the isoflavone from the sorbent to remove other undesired or contaminating materials. Alternatively, certain admixtures of alcohol and water that do not displace the isoflavone from the sorbent, or admixtures of an organic solvent and water that do not displace the isoflavone from the sorbent, can be used to remove other undesired or contaminating materials. Once the adsorbed material containing the isoflavone has been washed, the isoflavone can be released by using either an alcohol, an organic solvent, an admixture of alcohol and water that displaces the isoflavone from the sorbent, or admixtures of an organic solvent and water that displace the isoflavone from the sorbent.

Methods for the solid phase extraction of isoflavone biotransformation with anion exchange resins or sorbents have been described previously in the preceding section of the detailed description of the invention on solid phase extractions of isoflavone biotransformation products. As disclosed in Example 1 of U.S. Pat. No. 6,020,471, these methods are also effective in the solid phase extraction of isoflavone aglycones. It is therefore anticipated that anion exchange resin based extraction methods can also be used to effect isolation of isoflavones from the treated (i.e., converted) materials produced by the methods of this invention.

Other methods of solid phase extraction of isoflavones with non-polar or slightly polar resins are described in U.S. Pat. No. 4,428,876 and can be adapted to the isolation of isoflavones in the practice of this invention. Either slightly polar, acrylic ester based adsorbent resin or non-polar, styrene-vinylbenzene based adsorbent resin can be used in this method. Methanol or aqueous methanol is typically used to elute the isoflavones from the non-polar resin.

In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated.

### EXAMPLES

### Example 1

### Characterization of Isoflavone and Isoflavone Biotransformation Product Accumulation in S. erythraea and A. erythreum Fermentations

The first isoflavone related biotransformation that occurs during the erythromycin fermentation process involving S. erythrea is the hydrolysis of glucoside groups from the isoflavone core and the release of the free isoflavone aglycones (Hessler et al., Appl. Microbiol Biotechnol, 47:398-404,1997). However, this study indicated that the desired isoflavone aglycones did not accumulate in these fermentations. The object of this example was to characterize the isoflavone biotransformation products produced in these fermentations.

The erythromycin producing strain used for this example was the wild-type “white” Saccharopolyspora erythraea FL2267, which is the same strain as ATCC 11635 (American Type Culture Collection, Manassas, Va.). This strain is also referred to as FL300 in the experiments described herein. Spores of S. erythraea were produced on E20A agar (Reeves et al., Antimicrobiol. Agents Chemother, 46, 3892-3899, 2002). Seed cultures were prepared in CFMI broth (Carbohydrate-based Fermentation Medium, a variation of SCM broth described previously [12]. CFM1 is a medium designed for laboratory use where a soluble medium is desired for convenient analysis of growth and chemical analysis of erythromycin production. CFM1 per liter distilled water: Difco™ soluble starch, 60 g; Bacto™-soytone (Difco™), 20 g; CaCl2.2H2O (Sigma), 0.1 g; Bacto™-yeast extract (Difco™), 1.5 g; MOPS, 26.5 g; pH adjusted to 6.8 with 4N NaOH (Sigma, Saint Louis, Mo.). CFM1 lacks the glucose, vitamins and trace elements of modified SCM [12]; and has 60 g (4×) of soluble starch. Fermentations were performed in either CFM1 broth or OFM1 broth (Oil-based Fermentation Medium). OFM1 contains insoluble medium components and is meant to closely correlate to an industrial-type fermentation medium. OFM1 per liter: toasted nutrisoy flour (ADM, Decatur, Ill.), 22 g; Difco™ soluble starch, 15 g; CaCO3 powder (JT Baker, Phillipsburg, N.J.), 3 g; MgSO4.7H2O (JT Baker, Phillipsburg, N.J.), 0.5 g; FeSO4.7H2O (JT Baker, Phillipsburg, N.J.), 15 mg; Soy oil, 50 ml (ADM, Decatur, Ill.). Pigmentation and sporulation studies were conducted on R2T2 agar. R2T2 is R2T described in Weber et al. (J. Bacteriol., 172, 2372-2383, 1990) minus peptone.

Fermentations were performed in unbaffled 250-ml Erlenmeyer flasks with milk-filter closures. The flasks were incubated at 32.5° C.±0.2° C., and 65%±3% humidity on an Infors Multitron Shaker having 1-inch circular displacement. Seed cultures containing CFM1 broth were prepared on the same shaker and under the same growth conditions that the fermentations were performed. Seed cultures were inoculated from fresh spores prepared from E20A agar plates. Fermentations were inoculated with 1.25-ml of a seed culture in late logarithmic growth phase (40-45 h) into 25-ml of CFM1 or OFM1 broth. Consistency in the preparation of the seed culture was required for each strain to obtain and maintain optimum and reproducible fermentation performance from experiment to experiment. Fermentations were grown for 3-5 days; their volumes were then corrected for evaporation through the addition of water before being further analyzed. At the conclusion of the indicated fermentation period, the spent growth media was separated from the cells and extracted with ethyl acetate. The ethyl acetate extracts were then concentrated by evaporation to yield material for analysis by TLC or HPLC.

Three solvent systems were used for identification of the isoflavones by Thin-Layer Chromatography (TLC). First, chloroform:methanol:acetic acid (10:1:1) was used to separate isoflavone aglycones (genistein, daidzein). However, hexane:ethyl acetate:methanol (20:20:8) was used for the better resolution of the glycosylated isoflavones (“spots A, B and C”, genistin, daidzin). All reagents were HPLC grade. All samples for the TLC analyses were prepared as ethyl acetate solutions. As a standards 0.3 mg/ml ethyl acetate solutions of the pure isoflavones (Indofine, Hillsborough, N.J.) were used. The third solvent system used was chloroform:MeOH:water (80:20:2). For analytical TLC experiments, Silica Gel 60 F254 plates (Macherey-Nagel) with 0.2 mm thickness were used. Presence of the F254, a fluorescent indicator with a 254 nm excitation wavelength made possible identification of the isoflavone products that appeared as dark spots at 254 nm (UVP transilluminator).

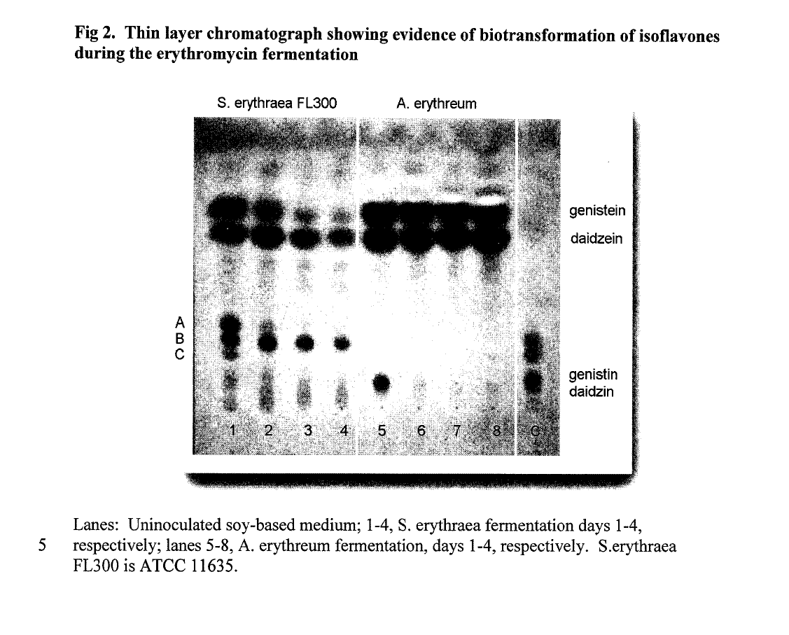

The aglycones, genistein and daidzein, remain in the fermentation only transiently and their disappearance coincides with appearance of two new spots on TLC (FIG. 2, spots A and B). Of the two new spots on TLC, spot A disappeared most rapidly, indicating a more rapid biotransformation or degradation. Analysis of the time course of isoflavone biotransformation by thin layer chromatography during the S. erythraea fermentation indicated that the spot shown as “A” (FIG. 2) was a biotransformation product of genistein, and spot “B” was a biotransformation product of daidzein.

In a fermentation biotransformation with another erythromycin-producing bacterium, Aeromicrobium erythreum, we observed that the first (desired) step of the conversion, genistin to genistein, occurred as rapidly as it did with S. erythraea, but the second (undesired) step, conversion of genistein, did not occur at all, even after four days growth. The two organisms fermentation extracts were compared side by side (FIG. 2) showing the striking difference in the two strains ability to biotransform isoflavones in fermentations. These experiments were performed in E29F broth, which is closely comparable to commercial production medium. A. erythreum is currently not used in industry because no high-producing strains are available; however, if this strain was used it would be predicted that it would have a large concentration of isoflavone aglycones in the spent fermentation broth. The A. erythreum fermentations thus show that isoflavones are chemically stable and can persist in E29F fermentation medium if biotransformation reaction enzymes are absent.

### Example 2

### Production, Purification, and Characterization of Spot A as Rhamnosyl-Genistein

Further evidence that spot A represented a biotransformation product of genistein was obtained when in vivo biotransformation reactions were performed in which pure genistein was added to S. erythraea cultures that were grown in a medium lacking soyflour and the same (i.e., comigrating) spot “A” observed in the original S. erythraea fermentations with soyflour appeared. To produce, purify and characterize the “spot A” biotransformation product of pure genistein, about 5 microliters of a dense spore suspension of S. erythraea was added to 40 ml of SCM broth in a 250 ml shake flask and incubated at 30° C. and 400 rpm (1 inch orbital displacement) for 2 days until cultures are thick and well grown. The entire 40 ml culture was transferred to a 2 L shake flask containing 100 ml of SCM and 100 mg of genistein (added directly to the medium from the vial), incubate the culture at 30° C. with shaking for 5 to 24 hrs. The broth was stored at 4° C. until all batches were ready for purification.

To purify the spot A isoflavone biotransformation product from the spent fermentation broth, the cells were first removed from the broth by centrifugation. The fermentation broth was then extracted twice with one-half volume of ethyl acetate and the solvent layers were combined and concentrated by evaporation under vacuum. The concentrated extracts were then applied to the top of a 12 g silica-gel flash chromatography column and processed on an Isco, Inc. Combi-Flash Companion Flash chromatography apparatus using a linear gradient elution method programmed to run from a starting condition of 25% hexane and 75% ethyl acetate to a finishing condition of 100% ethyl acetate over a 15 minute run. The individual fractions containing pure spot A isoflavone biotransformation product were identified by UV absorption profile and thin layer chromatography analysis. They were combined and dried completely in vacuo. The dried powder was then suspended in 80% ethanol, heated to 90° C. and dissolved completely, followed by cooling at 4° C. overnight. Crystals appeared overnight and were washed with cold ethanol and dried in vacuo.

The chemical analysis of spot “A” by mass spectral analysis, NMR analysis and X-ray Crystallographic analysis led to its clear structural identification as genistein 7-O-alpha-rhamnoside (FIG. 3).

### Example 3

### Cloning, Characterization and Inactivation of the Rhamnosyl-Transferase Gene of S. erythraea

The implication of the identity of spot A as genistein 7-O-alpha-rhamnoside was that the fermentation organism, S. erythraea must therefore harbor a gene coding for a rhamnosyltransferase that is capable of transferring a rhamnose group onto the genistein aglycone converting it to the rhamnosyl derivative. A further implication of this result was that if the gene coding for the isoflavone rhamnosyltransferase could be cloned and knocked out, then the unwanted biotransformation of the isoflavone aglycones could be stopped during the erythromycin fermentation, thus allowing the recovery of the isoflavone aglycones at the end of the fermentation.

To this end, the S. erythraea rht gene was cloned and analyzed. The DNA sequence of the S. erythraea rht gene was determined and analyzed by BLAST homology searches. The strongest homology of the S. erythraea rht gene was to a putative dTDP-rhamnosyl transferase from Mycobacterium leprae (gil|13092868|emb|CAC30261.1|) to which it showed a 60% sequence identity over a region of 270 bp. The same level of sequence identity was found to a putative dTDP-rhamnosyltransferase from Nocardia farcinica IFM 10152. (accession numbers gi|54018118|dbj|BAD59488.1|).

In order to knockout the rht gene of S. erythraea, an integrative plasmid was constructed which was designed to insert into the rht gene and inactivate the gene by insertional disruption. No transformants could be obtained with the plasmid construct designed to disrupt the rht gene, but in control experiments, many hundreds of transformants could be obtained with similar plasmid constructs that were designed to knockout another gene unrelated to the rht gene. These results suggested that rht might be a gene essential for the growth of S. erythraea and therefore could not be inactivated unless a replacement gene was transferred into the cell on the same plasmid that was used for the inactivation of the rht gene. When a replacement rhamnosyltransferase gene from a Corynebacterium species was included on the insertion plasmid, then it was found that insertional disruption of the S. erythraea rht gene could be achieved, however the disruption of rht did not stop the biotransformation of genistein to rhamnosylgenistein. It is believed that the failure to block biotransformation by replacement of the S. erythraea rht gene with the Corynebacterium rht gene was due to the ability of the Corynebacterium rht gene product to catalyze biotransformation of genistein to rhamnosylgenistein.

The conclusion of this series of experiments led to the development of another molecular genetic based strategy for stopping biotransformation of isoflavones in the erythromycin fermentation. These experiments, which attempted to block the first biotransformation reaction where the isoflavone glycosides from the soybean growth media were converted to isoflavones by inactivating endogenous S. erythraea beta-glucosidases and beta-galactosidases, also met with little success.

### Example 4

### Conversion of Isoflavone Biotransformation Products to Isoflavone Aglycones

Further analysis of thin layer chromatography plates and identification of highly polar UV-absorbing spots indicated that the final isoflavone biotransformation products may not be irreversibly degraded or modified as previously believed. To demonstrate that these final transformation products were actually reversibly modified biotransformation products, the isoflavone biotransformation products were obtained and treated in a conversion reaction. In this particular example, the conversion reaction consisted of acid and heat treatment. The products of this conversion reaction were then further purified and analyzed. Conversion of the isoflavone biotransformation products to isoflavones was thus demonstrated as described in detail below.

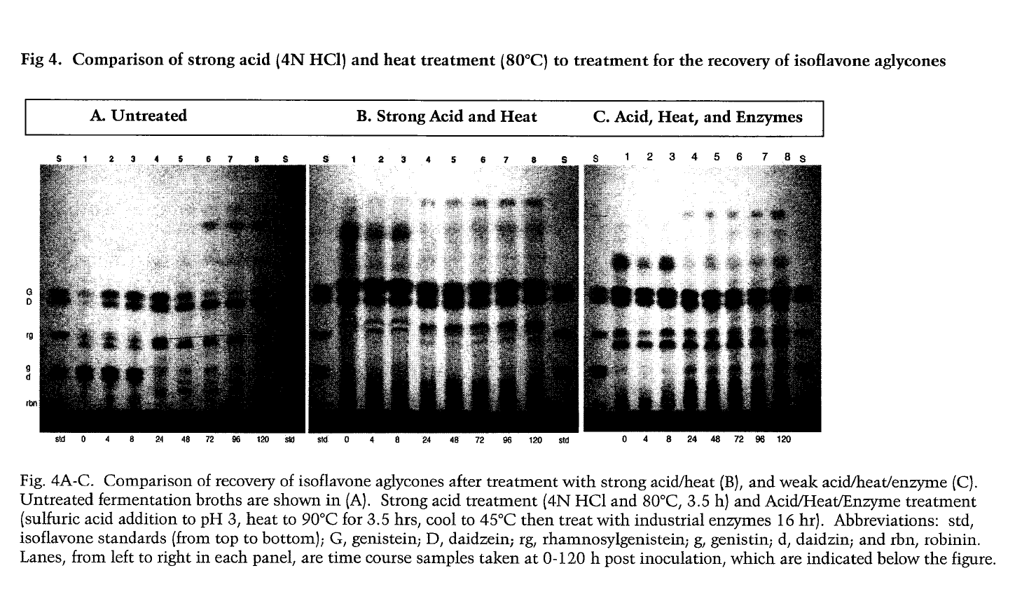

In brief, a fermentation was performed in modified OFM1 media containing 2× soyflour lacking soy oil. Soy oil was omitted and extra soyflour was added to simplify the analysis of the fermentation broths by thin layer chromatography. Two sets of samples were taken at each fermentation time point. In the first set of control samples, the cells were separated from the spent fermentation broths. The aqueous spent fermentation broths were extracted with an organic solvent (ethylacetate:n-butanol, 9:1), which was then separated from the aqueous phase, concentrated and analyzed by TLC (FIG. 4A). In the second set of samples, the cell-free spent fermentation broth was first subjected to heat and strong acid treatment prior to undergoing the same solvent extraction, concentration and analysis steps used to process the first set of control samples (FIG. 4B). Consequently, the only difference in the treatment and analysis of the first set of control samples and the second set of experimental samples was the treatment of the second set of experimental (i.e., treated) samples with heat and strong acid.

The strong acid and heat treatment was conducted as follows. To 500 ul of fermentation broth in a 15 ml polypropylene test tube with screw cap closure, 250 ul of concentrated (12 N) HCl was added. The tube was incubated at 80° C. for 3.5 h, cooled, and two ml of water was added to the acidified solution. The aqueous mixture was extracted once with 1.5 ml of ethylacetate:n-butanol (9:1). The solvent fraction was separated from the aqueous fraction, evaporated, and the remaining small residue was resuspended in 25 ul of ethyl acetate. Five ul of the resuspended product was spotted on thin layer silica gel plates which were then developed and visualized according to the method described in the previous Examples.

Samples taken prior to the start of the fermentation (0 hrs, FIG. 4A) show isoflavone glucosides, genistin and daidzin, to be the primary isoflavone constituents of the broth. As the fermentation progresses the glucosides are hydrolyzed to the aglycones which are then quickly converted to isoflavone rhamnosides so that both the isoflavone aglycones and the rhamnosides are visible at 24 hrs. By 48 hrs the genistein spot is significantly reduced, but the daidzein spot remains strong. The genistein rhamnoside spot disappears by 48 hrs. Other notable features are the appearance of minor bands at the lower Rf values on the TLC plate at 24 hrs which persist to the end of the fermentation.

Next the extracts of untreated fermentation broth (FIG. 4A) were compared to extracts of fermentation broth that were treated with strong acid (4N HCl) at high temperature (80° C., 3.5 hrs) (FIG. 4B). The most striking observation is that despite how the untreated samples vary from one time point to the next in the distribution of isoflavone constituents, the treated broth samples show remarkable homogeneity from one time point to the next. The primary result, as far as can be discerned by TLC, is that the isoflavone biotransformation products appear to be changed back to their aglycone form after the high temperature acid treatment. These results demonstrate that much of the isoflavone product at the end of the fermentation can be converted back into the aglycone form by high temperature acid treatment. Furthermore, the aglycone products can be recovered and isolated from other more polar materials that remain in the aqueous fraction from the treated spent broth by solvent (ethylacetate:n-butanol; 9:1) extraction.

### Example 5

### Combined Treatment of Spent Fermentation Broths with Acid, Heat and Enzymes also Converts Reversibly Modified Isoflavone Biotransformation Products into Isoflavones

U.S. Pat. No. 6,616,953 describes a milder acid treatment process using sulfuric acid in combination with heat and industrial enzyme treatments of spent fermentation broths of S. erythraea. This particular treatment method is of interest as it can be practically scaled up for operation on an industrial level. In the lab scale experiments described in this example, we demonstrate that the combined treatment of spent fermentation broths with acid, heat and enzymes is also highly effective in converting the biotransformation products in the spent fermentation broth into the desired isoflavone aglycones.

In brief, 3 ml of fermentation broth sample from S. erythraea were obtained as described in the previous examples. Concentrated sulfuric acid was then added (20-40 ul) to the 3 ml fermentation broth sample to lower the solution to pH 3. The solution was then heated to 90° C. for 3.5 hr, cooled to 45° C., at which point 30 ul of Validase® BG (beta-glucanase) and 30 ul of Crystalzyme® PML-MX (pectinase/cellulase) enzyme solutions were added. Validase® BG (an endo beta 1,4 glucanase) and Crystalzyme® PML-MX (a mixture consisting of cellulases, hemicellulases, pectinases, and arabinases) enzyme solutions were purchased from Valley Research, South Bend, Ind. The enzyme treatment was performed for 17 hrs before the solutions were solvent extracted as described in the previous examples for TLC analysis.

A comparison of the two methods for converting reversibly modified isoflavone biotransformation products is shown (FIG. 4B: strong acid and 4C: acid and enzymes). One significant difference between the two methods is the appearance of a new band just below rhanmosylgenistein (rg) but since it is seen in unfermented medium control (FIG. 4C, lane 1), it is not derived from a biotransformation product.

To test the ability of the Validase® BG (an endo beta 1,4 glucanase) and Crystalzyme® PML-MX (a mixture consisting of cellulases, hemicellulases, pectinases, and arabinases) enzyme solutions to perform the conversion reaction independently of one another, each enzyme preparation was tested individually and in combination. In brief, 3 ml of spent fermentation broth was brought to pH to 3 with sulfuric acid and heated for 3.5 hours at 90° C. After cooling to 45° C., either 1% Crystalzyme® PML-MX (C), 1% Validase® BG (V), or both (C+V) were added to the acid treated material. This was then incubated for 20 hrs and extracted with equal volumes of ethyl acetate:n-butanol (9:1), agitated for 1 min, and then centrifuged to separate the organic and aqueous phases. Organic phases were transferred to eppendorf tubes, dried in vacuo, and resuspended in 400 ul acetonitrile. About 380 ul of the supernatant was harvested and dried in vacuo. The material was then resuspended in 40 acetonitrile and spotted 5 ul/lane on TLC plates as described previously.

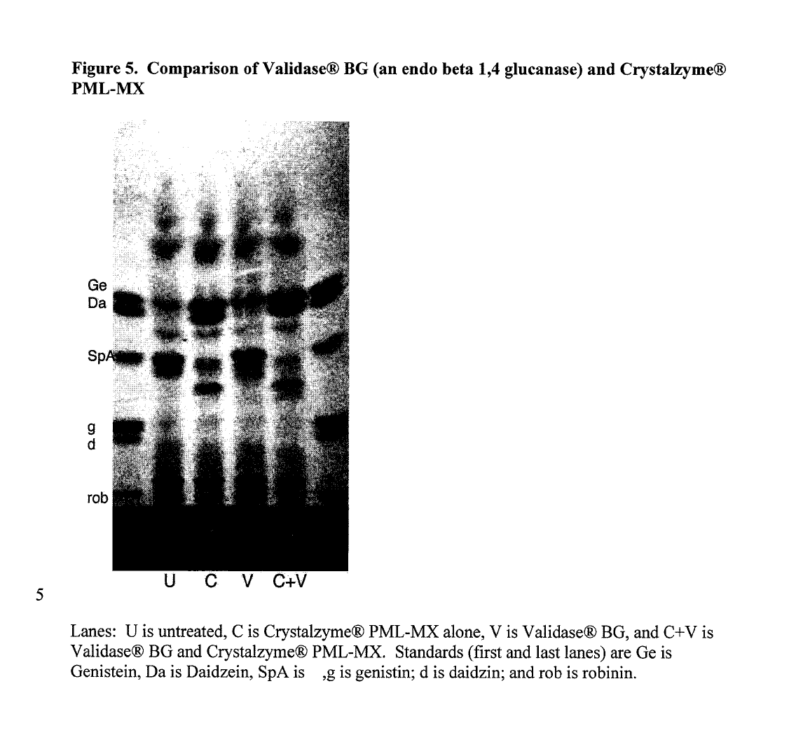

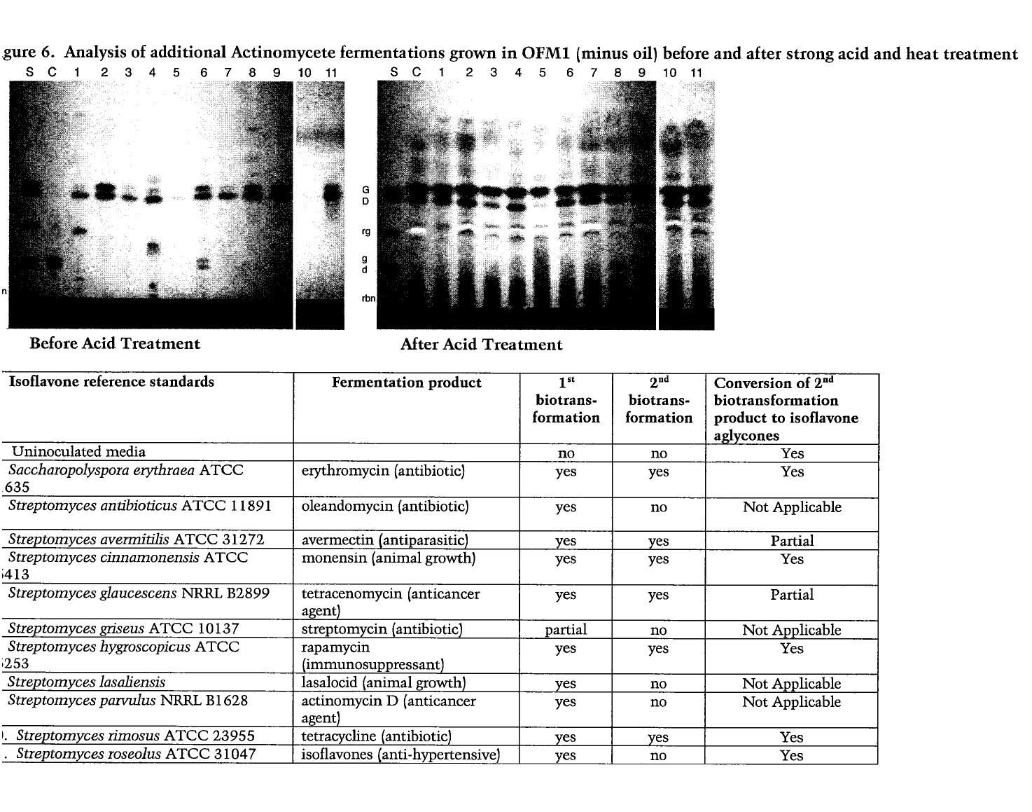

The results of the experiment shown in FIG. 5 demonstrate that Crystalzyme® PML-MX alone was sufficient to convert isoflavone biotransformation products to isoflavones. Crystalzyme-treated samples convert isoflavone glycosides to primarily the three compounds seen previously (genistein, daidzein and a spot mid-way between Spot A and genistin (compare lanes 2 and 4). Validase® BG treated samples (lane 3) looks similar to the untreated control (lane 1), except for one minor difference. Validase® BG, a bacterial 1,4-endo-beta glucanase, is thus insufficient to convert isoflavone biotransformation products to isoflavones in this method.

### Example 6

### Conversion of Reversibly Modified Isoflavones Produced by Other Streptomycetes

Ten other Streptomycetes of industrial importance were tested to demonstrate the production of reversibly modified isoflavone biotransformation products and their conversion to the desired isoflavone aglycones by acid and heat treatment. In these experiments, the erythromycin-producing organism, Saccharopolyspora erythraea was included as a positive control.

The results show that all of the Streptomycetes tested have the ability to perform the first isoflavone glucoside biotransformation reaction, namely, they are able to hydrolyze glucoside groups from isoflavone glucosides genistin and daidzin to form the isoflavone aglycones, genistein and daidzein. More importantly, the results also show that five out of ten of these other Streptomyces (numbers 3, 4, 5, 7, and 10, FIG. 6) appear to perform additional biotransformation reactions on the isoflavone aglycones, particularly genistein. This was determined by either the appearance of new spots on TLC, as for number 4 (S. cinnamonensis), or by simply the disappearance of either genistein or daidzein from the fermentation medium. Surprisingly, acid and heat treatment of three out of five of the biotransformers was sufficient to recover original levels of both of the isoflavone aglycones genistein and daidzein (numbers 4, 7, and 10). For Strains No. 3 (S. avermitilis), reduced but still substantial recovery of genistein was observed while recovery of daidzein was significantly reduced following acid and heat treatment. For Strain No. 5 (S. glaucescens), reduced recovery of genistein was observed and very little daidzein was recovered following acid and heat treatment. In conclusion, these results indicate that other Streptomyces can produce reversibly modified isoflavone biotransformation products that can be converted to isoflavones.

Five Streptomycetes catalyze the first desirable biotransformation reaction (i.e., hydrolysis of isoflavone glucosides in the fermentation media to form the isoflavone aglycones, genistein and daidzein) but fail to catalyze the subsequent biotransformation reactions that require conversion for recovery of isoflavone aglycones. These particular Streptomycetes with the desirable property of catalyzing first biotransformation reaction to form isoflavones (i.e., isoflavone aglycones) are Streptomyces roseolus, Streptomyces antibioticus, Streptomyces griseus, Streptomyces lasaliensis, and Streptomyces parvulus. This result indicates that isoflavones could be directly recovered from the spent fermentation beer produced when any one of these five Streptomycetes are fermented on a leguminous plant based substrate such as soy without having to perform conversion treatments. Production of isoflavones by Streptomyces roseolus in soy-based fermentations has previously been described in U.S. Pat. No. 5,554,519.

TABLE ONE
Summary of Isoflavone Biotransformation Product Formation by Various
Streptomycetes and Conversion to Isoflavone Aglycones

| | | | | Conversion of 2^{nd} |
|---|---|---|---|---|
| | | | | biotransformation |
| S. Isoflavone reference | | 1^{st} | 2^{nd} | product to |
| standards | Fermentation product | biotransformation | biotransformation | isoflavone aglycones |
| C. Uninoculated media | | no | no | Yes |
| 1. Saccharopolyspora | erythromycin (antibiotic) | yes | yes | Yes |
| erythraea ATCC 11635 | | | | |
| 2. Streptomyces antibioticus | oleandomycin (antibiotic) | yes | no | Not Applicable |
| ATCC 11891 | | | | |
| 3. Streptomyces avermitilis | avermectin (antiparasitic) | yes | yes | Partial |
| ATCC 31272 | | | | |
| 4. Streptomyces | monensin (animal growth) | yes | yes | Yes |
| cinnamonensis ATCC 15413 | | | | |
| 5. Streptomyces glaucescens | tetracenomycin (anticancer | yes | yes | Partial |
| NRRL B2899 | agent) | | | |
| 6. Streptomyces griseus | streptomycin (antibiotic) | partial | no | Not Applicable |
| ATCC 10137 | | | | |
| 7. Streptomyces | rapamycin | yes | yes | Yes |
| hygroscopicus ATCC 25253 | (immunosuppressant) | | | |
| 8. Streptomyces lasaliensis | lasalocid (animal growth) | yes | no | Not Applicable |
| 9. Streptomyces parvulus | actinomycin D (anticancer | yes | no | Not Applicable |
| NRRL B1628 | agent) | | | |
| 10. Streptomyces rimosus | tetracycline (antibiotic) | yes | yes | Yes |
| ATCC 23955 | | | | |
| 11. Streptomyces roseolus | isoflavones (anti- | yes | no | Yes |
| ATCC 31047 | hypertensive) | | | |

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

Each and every patent and non-patent literature reference cited herein is hereby specifically incorporated by reference in its entirety.

What is claimed is:

1. A method for producing an isoflavone in a fermentation process, comprising the steps of:
a. growing an actinomycete selected from the group consisting of Streptomyces and Saccharopolyspora, in a growth medium comprising material from a leguminous plant;
b. obtaining an isoflavone biotransformation product from a spent growth medium from step (a), wherein said isoflavone biotransformation product is capable of being converted into an isoflavone;
c. converting said isoflavone biotransformation product from step (b) to an isoflavone;
d. isolating said isoflavone from step (c), thereby producing an isoflavone in a fermentation process.

2. The method of claim 1, wherein said isoflavone is genistein or daidzein.

3. The method of claim 1, wherein said actinomycete is selected from the group consisting of Streptomyces rimosus, Streptomyces fradiae, Streptomyces hygroscopicus, Streptomyces cinnamonensis, Streptomyces peucetius, Saccharopolyspora erythraea, Streptomyces avermitilis, Streptomyces glaucescens, and Streptomyces roseolous.

4. The method of claim 3, wherein said actinomycete is Saccharopolyspora erythraea.

5. The method of claim 1, wherein said isoflavone biotransformation product is not an irreversibly modified isoflavone.

6. The method of claim 5, wherein said irreversibly modified isoflavone is an isoflavone that has been oxidized, reduced, or hydrolyzed.

7. The method of claim 5, wherein said irreversibly modified isoflavone is a hydroxylated, methoxylated, methylated or chlorinated isoflavone.

8. The method of claim 1, wherein said actinomycete glycosylates an isoflavone in step (a).

9. The method of claim 8, wherein said actinomycete rhamnosylates an isoflavone in step (a).

10. The method of claim 1, wherein said actinomycete esterifies an isoflavone in step (a).

11. The method of claim 1, wherein said leguminous plant is selected from the group consisting of soybean (Glycine max), lupine (Lupinus species), fava bean, (Vicia faba), bean (Phaseolus species), clover (Trifolium species), lentil (Lens species), Baptisia (Baptisia species), kudzu (Pueraria species), pea (Pisum species), Vigna species, garbanzo bean (Cicer species), alfalfa (Medicago species), and psoralea (Psoralea corylifolia).

12. The method of claim 1, wherein said leguminous plant is soybean (Glycine max).

13. The method of claim 1, wherein said isoflavone biotransformation product is obtained in step (b) by extracting said growth medium from step (a) with a polar or non-polar organic solvent which is immiscible with water to obtain a solvent fraction and an aqueous fraction, wherein said aqueous fraction contains said isoflavone biotransformation product.

14. The method of claim 13, wherein said polar or non-polar organic solvent is a C₂-C₁₀ organic solvent.

15. The method of claim 14, wherein said polar or non-polar organic solvent is selected from the group consisting of 1-butanol, 2-butanol, t-butanol, pentanol, hexanol, heptanol, octanol, ether, ethyl acetate, tetrahydrofuran, hexane, heptane, octane, isohexane, diethylether, methyl ethyl ketone, diisopropylether propanol, isopropyl alcohol, isobutyl alcohol, butanol, ethyl acetate, acetonitrile, acetone, methylene chloride, chloroform, carbon tetrachloride, and mixtures thereof.

16. The method of claim 1, wherein said isoflavone biotransformation product is obtained in step (b) by capturing said isoflavone biotransformation product from said growth medium of step (a) on a solid phase sorbent and eluting said isoflavone biotransformation product from said solid phase sorbent.

17. The method of claim 14, wherein said solid phase sorbent is selected from the group consisting of a non-polar polystyrene sorbent, a C18 sorbant, a divinylbenzene sorbant, an anion exchange sorbent, a polystyrene-divinylbenzene sorbant and a modified divinylbenzene sorbent.

18. The method of claim 1, wherein said isoflavone biotransformation product is converted to an isoflavone in step (c) by treating said isoflavone biotransformation product from step (b) with a sufficient amount of an acid and a sufficient degree of heat to result in conversion of said isoflavone biotransformation product to an isoflavone.

19. The method of claim 18, wherein said acid is selected from the group consisting of boric acid, benzoic acid, butyric acid, carbonic acid, citric acid, hydrobromic acid, hydrochloric acid, hydrofluoric acid, hydroiodic acid, lactic acid, malic acid, mandelic acid, nitric acid, propionic acid, sulfuric acid, oxalic acid, perchloric acid, phosphoric acid, phosphonic acid, pyrophosphoric acid, pyruvic acid, valeric acid, acetic acid and formic acid.

20. The method of claim 18, wherein said isoflavone biotransformation product is treated with about 3N to 6N HCl at a temperature of about 50° C. to 80° C. for about 3 hours.

21. The method of claim 20, wherein said isoflavone biotransformation product is treated with about 3N to 4 N HCl at about 80° C. for about 3.5 hours.

22. The method of claim 1, further comprising the step of concentrating the isoflavone biotransformation product of step (b) prior to conversion of said isoflavone biotransformation product in step (c).

23. The method of claim 22, wherein said concentration is effected by performing a solid phase extraction of the isoflavone biotransformation product of step (b) and recovering said isoflavone biotransformation product in a concentrated form.

24. The method of claim 22, wherein said solid phase extraction is effected with a sorbent selected from the group consisting of non-polar polystrenes, C18, divinylbenzene, polystyrene-divinylbenzene and modified divinylbenzene.

25. The method of claim 22, wherein said concentration is effected by extracting said growth medium from step (a) with a polar or non-polar organic solvent which is immiscible in water to obtain a solvent fraction and an aqueous fraction, increasing the acidity of said aqueous fraction of step (b) to about pH 3, extracting the acidified fraction of the preceding step with a solvent, evaporating said solvent and recovering said isoflavone biotransformation product in a concentrated form.

26. The method of claim 1, wherein said isoflavone biotransformation product of steps (b) and (c) is a glycosylated or an esterified isoflavone.

27. The method of claim 1, wherein said isoflavone biotransformation product is converted to an isoflavone in step (c) by a first step comprising a treatment of said isoflavone biotransformation product from step (b) with an acid and heat followed by a second step comprising a treatment of said acid and heat treated biotransformation product from the preceding step with at least one enzyme selected from the group consisting of a cellulase, a hemicellulase, a pectinase, an arabinosidase, or any combination thereof, wherein an amount of acid and heat in said first step and an amount of enzyme in said second step sufficient to result in conversion of said isoflavone biotransformation product to an isoflavone are used.

28. The method of claim 27, wherein said acid in said first step is selected from the group consisting of boric acid, benzoic acid, butyric acid, carbonic acid, citric acid, hydrobromic acid, hydrochloric acid, hydrofluoric acid, hydroiodic acid, lactic acid, malic acid, mandelic acid, nitric acid, propionic acid, sulfuric acid, oxalic acid, perchloric acid, phosphoric acid, phosphonic acid, pyrophosphoric acid, pyruvic acid, valeric acid, acetic acid and formic acid.

29. The method of claim 27, wherein said isoflavone biotransformation product is treated in said first step with acid at a temperature from about 80° C. to about 100° C.

30. The method of claim 27, wherein said isoflavone biotransformation product is treated in said first step with acid sufficient to obtain a pH of about 3.5-5.0.

31. The method of claim 27, wherein a cellulase is used in said second step.

32. The method of claim 31, wherein a pectinase is used in said second step.

33. The method of claim 32, wherein mixture of cellulase, a hemi-cellulase, a pectinase and an arabinosidase is used in said second step.

34. The method of claim 27, further comprising the step of cooling the acid and heat treated biotransformation product from said first step to a temperature from about 40° C. to about 60° C. before adding at least one enzyme in a subsequent step.

35. The method of claim 1, wherein isolation of said isoflavone in step (d) is effected by extracting said isoflavone from step (c) from an aqueous phase with a polar or non-polar solvent that is immiscible in water, wherein said isoflavone is recovered in said solvent.

36. The method of claim 35, wherein said solvent is selected from the group consisting of propanol, isopropyl alcohol, isobutyl alcohol, butanol, ethyl acetate, acetonitrile, acetone, methylene chloride, chloroform, carbon tetrachloride, and mixtures thereof.

37. The method of claim 1, wherein isolation of said isoflavone in step (d) is effected by performing a solid phase extraction of said isoflavone from step (c).

38. The method of claim 37, wherein said solid phase extraction is effected with a sorbent selected from the group consisting of C18, divinylbenzene, polystyrene-divinylbenzene, non-polar polystyrene, anion exchange resin, and modified divinylbenzene.

39. The method of claim 38, wherein said sorbent is an anion exchange resin and wherein isoflavone is released by treating said resin with an alcohol, an organic solvent, an admixture of alcohol and water, or an admixture of an organic solvent and water.

40. The method of claim 1, wherein said isolated isoflavone of step (d) is substantially free of saccharides.

41. The method of claim 1, wherein said fermentation process is a process for producing a drug or a drug precursor.

42. The method of claim 41, wherein said drug is antibiotic, an animal growth modulator, an anti-cancer agent, an immunosuppressant, an anti-hypertensive agent, or an anti-parasitic agent.

43. The method of claim 41, wherein said drug or a drug precursor is selected from the group consisting of oleandomycin, actinomycin, avermectin, lasalocid, tetracenomycin, tetracycline, oxytetracycline, tylosin, rapamycin, daunorubicin, rifamycin, monensin and erythromycin.

44. The method of claim 41, wherein said drug or drug precursor is erythromycin.

45. The method of claim 1, wherein at least 50% of said isoflavone biotransformation product obtained in step (b) is converted to an isoflavone in step(c).

46. The method of claim 45, wherein at least 70% of said isoflavone biotransformation product obtained in step (b) is converted to an isoflavone in step(c).

47. The method of claim 45, wherein at least 95% of said isoflavone biotransformation product obtained in step (b) is converted to an isoflavone in step(c).

48. A method for producing an isoflavone in a fermentation process, comprising the steps of:
a. growing Saccharopolyspora erythrea in a growth medium comprising material from a soybean plant;
b. obtaining an isoflavone biotransformation product from a spent growth medium from step (a), wherein said isoflavone biotransformation product is capable of being converted into an isoflavone;
c. converting said isoflavone biotransformation product from step (b) to an isoflavone;
d. isolating said isoflavone from step (c), thereby producing an isoflavone in a fermentation process.

49. The method of claim 48, wherein said isoflavone is genistein or daidzein.

50. The method of claim 48, wherein said isoflavone biotransformation product of steps (b) and (c) is a glycosylated or an esterified isoflavone.

51. The method of claim 48, wherein Saccharopolyspora erythraea ATCC11635 is used in step (a).

52. The method of claim 48, wherein said isoflavone biotransformation product is converted to an isoflavone in step (c) by a first step comprising a treatment of said isoflavone biotransformation product from step (b) with an acid and heat followed by a second step comprising a treatment of said acid and heat treated biotransformation product from the preceding step with at least one enzyme selected from the group consisting of a cellulase, a hemicellulase, a pectinase, an arabinosidase, or any combination thereof, wherein an amount of acid and heat in said first step and an amount of enzyme in said second step sufficient to result in conversion of said isoflavone biotransformation product to an isoflavone are used.

53. The method of claim 52, wherein a cellulase is used in said second step.

54. The method of claim 52, wherein a pectinase is used in said second step.

55. The method of claim 52, wherein mixture of cellulase, a hemi-cellulase, a pectinase and an arabinosidase is used in said second step.

56. The method of claim 48, wherein said isoflavone biotransformation product is converted to an isoflavone in step (c) by treating said isoflavone biotransformation product from step (b) with a sufficient amount of an acid and a sufficient degree of heat to result in conversion of said isoflavone biotransformation product to an isoflavone.

57. The method of claim 52, wherein said acid is selected from the group consisting of boric acid, benzoic acid, butyric acid, carbonic acid, citric acid, hydrobromic acid, hydrochloric acid, hydrofluoric acid, hydroiodic acid, lactic acid, malic acid, mandelic acid, nitric acid, propionic acid, sulfuric acid, oxalic acid, perchloric acid, phosphoric acid, phosphonic acid, pyrophosphoric acid, pyruvic acid, valeric acid, acetic acid and formic acid.

58. The method of claim 48, wherein isolation of said isoflavone in step (d) is effected by extracting said isoflavone from step (c) from an aqueous phase with a polar or non-polar organic solvent that is immiscible in water.

59. The method of claim 48, wherein isolation of said isoflavone in step (d) is effected by performing a solid phase extraction of said isoflavone from step (c).

60. A method for producing an isoflavone in a fermentation process, comprising the steps of:
a. growing a Streptomycete selected from the group consisting of Streptomyces antibioticus, Streptomyces griseus, Streptomyces lasaliensis, and Streptomyces parvulus in a growth medium comprising material from a leguminous plant;
b. obtaining an isoflavone from a spent growth medium from step (a);
c. isolating said isoflavone from step (b),
thereby producing an isoflavone in a fermentation process.
